(19) United States
(12) Patent Application Publication
(10) Pub. No.: US 2007/0299088 A1
(43) Pub. Date: Dec. 27, 2007

(54) Novel Uses of 2-Phenyl-Substituted Imidazotriazinone Derivatives

(75) Inventor: Helmut Haning, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 11/659,624

(22) PCT Filed: Jul. 23, 2005
(86) PCT No.: PCT/EP05/08057

§ 371 Date: Sep. 5, 2007

(30) Foreign Application Priority Data
Aug. 6, 2004 (DE) 10 2004 038 328.6

(51) Int. Cl. A61K 31/522; A61P 1/16; A61P 15/08; A61P 17/00; A61P 17/14; A61P 25/06; A61P 25/08; A61P 35/02; A61P 43/00; A61P 9/10

(52) U.S. Cl. 514/259.5

(57) ABSTRACT

The invention relates to the use of PDE 5 inhibitors, and especially of known 2-phenyl-substituted imidazotriazinone derivatives for producing medicaments for the treatment of symptoms that can be treated by increasing cGMP levels in certain tissues, such as acute myocardial infarction and damage caused by reperfusion, various symptoms in the female and male reproductive system and urogenital tract, gastrointestinal diseases, damage caused by diabetes, and liver failure.

The present invention relates to the use of PDE 5 inhibitors generally and in particular of known 2-phenyl-substituted imidazotriazinone derivatives for manufacturing medicaments for the treatment of pathological states which are treatable by raising cGMP levels in certain tissues, such as, for example, of acute myocardial infarction and reperfusion damage, of various pathological states of the female and male reproductive system and urogenital tract, of gastrointestinal disorders, diabetic damage and of renal failure.

The cyclic nucleotide cGMP (cyclic guanosine monophosphate) is one of the most important intracellular messengers and is metabolized by certain phosphodiesterases (PDEs), in particular the isoenzyme PDE 5, [Drugs Fut. 26, 153-162 (2001)]. PDE 5 occurs in particular in vascular smooth muscle cell tissue, and less in the kidney, lung and the blood platelets. Owing to their vasorelaxant effect, PDE 5 inhibitors are proposed for the treatment of angina and high blood pressure, but mainly for the treatment of erectile dysfunction.

WO 99/24433 describes 2-phenyl-substituted imidazotriazinones, their cGMP-PDE-inhibiting effect and their use for the treatment of vascular disorders, in particular for the treatment of erectile dysfunction. WO 02/089808 and WO 03/011262 disclose uses of 2-phenyl-substituted imidazotriazinones.

The literature at present describes 11 phosphodiesterases differing in specificity in relation to the cyclic nucleotides cAMP and cGMP [cf. Fawcett et al., Proc. Nat. Acad. Sci. 97 (7), 3072-3077 (2000)]. Cyclic guanosine 3′,5′-monophosphate-metabolizing phosphodiesterases (cGMP-PDEs) are PDE 1, 2, 5, 6, 9, 10 and 11. The mentioned 2-phenyl-substituted imidazotriazinones used according to the invention are potent inhibitors of phosphodiesterase 5. Differential expression of the phosphodiesterases in different cells, tissues and organs, as well as the differential subcellular localization of these enzymes, make it possible in conjunction with selective inhibitors to raise selectively the cGMP concentration in specific cells, tissues and organs and thus allow various cGMP-regulated processes to be addressed, so that PDE 5 inhibitors can be used therapeutically in a number of pathological states which can be influenced by raising the cGMP level.

PDE 5 inhibitors preferred in this connection are those which, in the assay detailed hereinafter, inhibit PDE 5 with an IC₅₀ of less than 1 μM, preferably of less than 0.1 μM.

The PDE 5 inhibitors used according to the invention are preferably also selective in relation to cAMP PDEs, in particular in relation to PDE 4. It is particularly preferred for the inhibition of PDE 5 to be at least ten times greater.

Compounds having an inhibitory effect on cGMP PDEs are described for example in the following publications: EP-A-0 201 188, EP-A-0 214 708, EP-A-0 293 063, EP-A-0 319 050, EP-A-0 347 027, EP-A-0 347 146, EP-A-0 349 239, EP-A-0 351 058, EP-A-0 352 960, EP-A-0 371 731, EP-A-0 395 328, EP-A-0 400 799, EP-A-0 428 268, EP-A-0 442 204, EP-A-0 463 756, EP-A-0 526 004, EP-A-0 579 496, EP-A-0 607 439, EP-A-0 640 599, EP-A-0 669 324, EP-A-0 686 625, EP-A-0 722 936, U.S. Pat. No. 4,060,615, U.S. Pat. No. 5,294,612, WO 91/19717, WO 94/19351, WO 94/22855, WO 96/32379, WO 97/03070, JP-A-05222000 (CAPLUS 1994, 191719).

Compounds having an inhibitory effect on the cGMP-specific PDE (corresponds to PDE 5) are described for example in the following publications: EP-A-0 636 626, EP-A-0 668 280, EP-A-0 722 937, EP-A-0 722 943, EP-A-0 722 944, EP-A-0 758 653, EP-A-0 995 750, EP-A-0 995 751, EP A-1 092 719, WO 94/28902, WO 95/19978, WO 96/16657, WO 96/28159, WO 96/28429, WO 98/49166, WO 99/24433, WO 99/67244, WO 00/78767, WO 01/12608, WO 01/18004, WO 01/19369, WO 01/19802, WO 01/21620, WO 01/27105, J. Med. Chem. 39, 1635-1644 (1996), J. Med. Chem. 43, 1257-1263 (2000), Drugs Fut. 26, 153-162 (2001).

The disclosure of these publications, in particular the compounds disclosed therein, is incorporated herein by reference.

One aspect of the present invention relates to the use of compounds of the general formula (I) 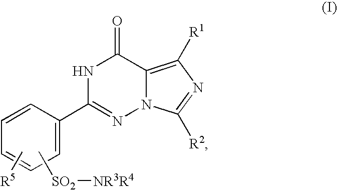
in which
R¹ is methyl or ethyl,
R² is ethyl or propyl,
R³ and R⁴ are identical or different and are a straight-chain or branched alkyl chain having up to 5 carbon atoms which is optionally substituted up to twice identically or differently by hydroxy or methoxy,
or
R³ and R⁴ together with the nitrogen atom form a piperidinyl, morpholinyl, thiomorpholinyl ring or a radical of the formula 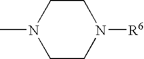
   in which
   R⁶ is hydrogen, formyl, acyl or alkoxycarbonyl having in each case up to 3 carbon atoms,
      or
      is straight-chain or branched alkyl having up to 3 carbon atoms which is optionally substituted once to twice, identically or differently, by hydroxy, carboxyl, straight-chain or branched alkoxy or alkoxycarbonyl having in each case up to 3 carbon atoms or by groups of the formulae —(CO)_{f}—NR⁷R⁸ or —P(O)(OR⁹)(OR¹⁰),
      in which
      f is a number 0 or 1,
      R⁷ and R⁸ are identical or different and are hydrogen or methyl,
      R⁹ and R¹⁰ are identical or different and are hydrogen, methyl or ethyl,
   or
   R⁶ is cyclopentyl,
and the heterocycles mentioned under R³ and R⁴ and formed together with the nitrogen atom are optionally substituted once to twice, identically or differently, optionally also geminally, by hydroxy, formyl, carboxyl, acyl or alkoxycarbonyl having in each case up to 3 carbon atoms or groups of the formulae —P(O)(OR¹¹)(OR¹²) or —(CO)ᵢ—NR¹³R¹⁴,
   in which
   R¹¹ and R¹² are identical or different and are hydrogen, methyl or ethyl,
   i is a number 0 or 1,
   and
   R¹³ and R¹⁴ are identical or different and are hydrogen or methyl,
and/or the heterocycles mentioned under R³ and R⁴ and formed together with the nitrogen atom are optionally substituted by straight-chain or branched alkyl having up to 3 carbon atoms, which is optionally substituted once to twice, identically or differently, by hydroxy, carboxyl or by a radical of the formula —P(O)OR¹⁵OR¹⁶,
   in which
   R¹⁵ and R¹⁶ are identical or different and are hydrogen, methyl or ethyl,
and/or the heterocycles mentioned under R³ and R⁴ and formed together with the nitrogen atom are optionally substituted by N-linked piperidinyl or pyrrolidinyl,
and
R⁵ is ethoxy or propoxy,
and the salts and solvates thereof and the solvates of the salts,
   for the treatment of cardiac ischemia, for achieving or improving a preconditioning effect, for the treatment of an acute myocardial infarction and of reperfusion damage, specifically following a myocardial infarction, for the treatment of male infertility, of Raynaud's syndrome, of intermittent claudication, of Peyronie's disease, for the treatment of fibrotic disorders, of arteriosclerosis, for improving sperm motility, for the treatment of depression, leukemia (e.g. of chronic lymphocytic leukemia), for the treatment of priapism, for the treatment of platelet adhesion and aggregation associated with renal ischemia, for supporting and promoting liver regeneration following surgical resection of the liver or associated with liver cancer, for inhibiting the contraction of esophageal muscles (e.g. associated with nutcracker esophagus or esophagospasms), for the treatment of achalasia, permature labor, female infertility and dysmenorrhea, for the treatment of liver disorders such as, for example, cirrhosis of the liver, portal hypertension, for the treatment of lupus, hypertensive systemic lupus erythematosus, scleroderma, for the treatment of multiple sclerosis, rheumatoid arthritis, allergy, autoimmune diseases, osteoporosis, cachexia, polycystic ovary syndrome, inflammatory bowel diseases such as, for example, Crohn's disease and ulcerative colitis, diabetic gangrene, diabetic arthropathy, diabetic glomerulosclerosis, diabetic dermatopathy, diabetic cataract, hyperlipidemia and dyslipidemia, for promoting growth and improving survival of oocytes, zygotes, embryos or fetuses, for increasing the weight of premature babies, for increasing milk production in mammals, specifically in humans, for the treatment of migraine, incontinence, acute and chronic renal failure, of glomerular disease, of nephritis, tubulointerstitial disorders, glomuleropathy, hair loss, pancreatitis, amnesia, disturbances of consciousness, autism, speech disturbances, Lennox syndrome and epilepsy.

The compounds used according to the invention may, depending on their structure, exist in stereoisomeric forms (enantiomers, diastereomers). The invention therefore includes the use of the enantiomers and diastereomers and respective mixtures thereof. The stereoisomerically pure constituents can be isolated from such mixtures of enantiomers and/or diastereomers in a known manner.

If the compounds used according to the invention can occur in tautomeric forms, the present invention includes all tautomeric forms.

Salts preferred in the context of the present invention are physiologically acceptable salts of the compounds used according to the invention. Also included are salts which are themselves unsuitable for pharmaceutical applications but can be used for example to isolate or purify the compounds used according to the invention.

Physiologically acceptable salts of the compounds used according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds used according to the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, prokaine, dibenzylamine, N-methylmorpholine, arginine, lysine, ethylenediamine and N-methylpiperidine.

Solvates refer in the context of the invention to those forms of the compounds used according to the invention which form in the solid or liquid state a complex by coordination with solvent molecules. Hydrates are a specific type of solvates in which the coordination takes place with water. Hydrates are preferred as solvates in the context of the present invention. Hydrates can be prepared for example by crystallizing the relevant compound from water or a hydrous solvent.

The present invention also includes in addition prodrugs of the compounds used according to the invention. The term “prodrugs” includes compounds which themselves may be biologically active or inactive but are converted (for example by metabolism or hydrolysis) during their residence time in the body into the compounds used according to the invention.

In the context of the present invention, the substituents have the following meaning unless specified otherwise:

An acyl radical having 1 to 3 carbon atoms is in the context of the invention for example formyl, acetyl or propionyl.

A straight-chain or branched alkoxy radical having 1 to 3 carbon atoms is in the context of the invention for example methoxy, ethoxy, n-propoxy or isopropoxy.

An alkoxycarbonyl radical having 1 to 3 carbon atoms is in the context of the invention for example methoxycarbonyl, ethoxycarbonyl or propoxycarbonyl.

A straight-chain or branched alkyl radical having 1 to 5 or 1 to 3 carbon atoms is in the context of the invention for example methyl, ethyl, n-propyl, isopropyl, tert-butyl or n-pentyl. Straight-chain or branched alkyl radicals having 1 to 4 or 1 to 3 carbon atoms are preferred.

A further embodiment of the invention relates to the use according to the invention of compounds of the general formula (I) in which the radicals R⁵ and —SO₂NR³R⁴ are positioned parallel to one another on the phenyl ring, and R¹, R², R³, R⁴ and R⁵ each have the meaning indicated above.

A further embodiment of the invention relates to the use according to the invention of compounds of the general formula (Ia)

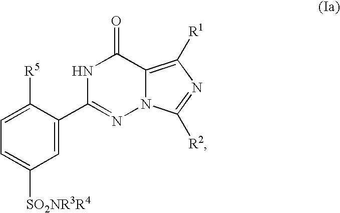

in which R¹, R², R³, R⁴ and R⁵ each have the meaning indicated above,

and the salts and solvates thereof and the solvates of the salts.

The use according to the invention of the following compounds is preferred:

2-[2-Ethoxy-5-(4-methylpiperazine-1-sulfonyl)-phenyl]-5,7-dimethyl-3H-imidazo[5,1-f][1,2,4]-triazin-4-one;

2-[2-Ethoxy-5-(4-hydroxyethylpiperazine-1-sulfonyl)-phenyl]-5,7-dimethyl-3H-imidazo[5,1-f]-[1,2,4]triazin-4-one;

2-[2-Ethoxy-5-(4-hydroxypiperidine-1-sulfonyl)-phenyl]-5,7-dimethyl-3H-imidazo[5,1-f][1,2,4]-triazin-4-one;

2-[2-Ethoxy-5-(4-hydroxymethylpiperidine-1-sulfonyl)-phenyl]-5,7-dimethyl-3H-imidazo[5,1-f]-[1,2,4]triazin-4-one;

2-[2-Ethoxy-5-(3-hydroxypyrrolidine-1-sulfonyl)-phenyl]-5,7-dimethyl-3H-imidazo[5,1-f][1,2,4]-triazin-4-one;

4-Ethoxy-N-ethyl-N-(2-hydroxyethyl)-3-(5,7-dimethyl-4-oxo-3,4-dihydro-imidazo[5,1-f][1,2,4]-triazin-2-yl)benzenesulfonamide;

N,N-Diethyl-4-ethoxy-3-(5,7-dimethyl-4-oxo-3,4-dihydroimidazo[5,1-f][1,2,4]triazin-2-yl)-benzenesulfonamide;

2-[2-Ethoxy-5-(4-(2-pyrimidinyl)-piperazine-1-sulfonyl)-phenyl]-5,7-dimethyl-3H-imidazo-[5,1-f]-[1,2,4]triazin-4-one;

2-[2-Ethoxy-5-(morpholine-4-sulfonyl)-phenyl]-5,7-dimethyl-3H-imidazo[5,1-f][1,2,4]triazin-4-one;

2-[2-Ethoxy-5-(1,4-dioxa-6-azaspiro[4.4]nonane-6-sulfonyl)-phenyl]-5,7-dimethyl-3H-imidazo[5,1-f][1,2,4]triazin-4-one;

N,N-Bis-(2-Methoxyethyl)-4-ethoxy-3-(5,7-dimethyl-4-oxo-3,4-dihydroimidazo[5,1-f][1,2,4]-triazin-2-yl)-benzenesulfonamide;

N-(3-Isoxazolyl)-4-ethoxy-3-(5,7-dimethyl-4-oxo-3,4-dihydroimidazo[5,1-f][1,2,4]triazin-2-yl)-benzenesulfonamide;

2-[2-Ethoxy-5-(2-tert.-butoxycarbonylaminomethylmorpholine-4-sulfonyl)-phenyl]-5,7-dimethyl-3H-imidazo[5,1-f][1,2,4]triazin-4-one;

2-[2-Ethoxy-5-(4-phenylpiperazine-1-sulfonyl)-phenyl]-5,7-dimethyl-3H-imidazo[5,1-f][1,2,4]-triazin-4-one;

2-[2-Ethoxy-5-(3-hydroxy-3-methoxymethylpyrrolidine-1-sulfonyl)-phenyl]-5,7-dimethyl-3H-imidazo[5,1-f][1,2,4]triazin-4-one;

2-[2-Ethoxy-5-(4-methylpiperazine-1-sulfonyl)-phenyl]-5-methyl-7-propyl-3H-imidazo[5,1-f]-[1,2,4]triazin4-one;

2-[2-Ethoxy-5-(4-methylpiperazine-1-sulfonyl)-phenyl]-5-methyl-7-propyl-3H-imidazo[5,1-f]-[1,2,4]triazin-4-one lactate;

2-[2-Ethoxy-5-(4-methylpiperazine-1-sulfonyl)-phenyl]-5-methyl-7-propyl-3H-imidazo[5,1-f]-[1,2,4]triazin-4-one hydrochloride;

2-[2-Ethoxy-5-(4-ethylpiperazine-1-sulfonyl)-phenyl]-5-methyl-7-propyl-3H-imidazo[5,1-f][1,2,4]-triazin-4-one;

2-[2-Ethoxy-5-(4-ethylpiperazine-1-sulfonyl)-phenyl]-5-methyl-7-propyl-3H-imidazo[5,1-f][1,2,4]-triazin-4-one hydrochloride;

2-[2-Ethoxy-5-(4-methyl-1-amino-piperazine-1-sulfonyl)-phenyl]-5-methyl-7-propyl-3H-imidazo-[5,1-f][1,2,4]triazin-4-one;

2-[2-Ethoxy-5-(4-hydroxyethyl-1-amino-piperazine-1-sulfonyl)-phenyl]-5-methyl-7-propyl-3H-imidazo[5,1-f][1,2,4]triazin-4-one;

N,N-Bishydroxyethylaminoethyl-4-ethoxy-3-(5-methyl-4-oxo-7-propyl-3,4-dihydro-imidazo[5,1-f]-[1,2,4]triazin-2-yl)benzenesulfonamide;

2-[2-Ethoxy-5-(4-dimethoxyphosphorylmethyl-piperazine-1-sulfonyl)-phenyl]-5-methyl-7-propyl-3H-imidazo[5,1-f][1,2,4]triazin-4-one;

2-[2-Ethoxy-5-(4-diethoxyphosphorylmethyl-piperidine-1-sulfonyl)-phenyl]-5-methyl-7-propyl-3H-imidazo[5,1-f][1,2,4]triazin-4-one;

2-[2-Ethoxy-5-(4-hydroxypiperidine-1-sulfonyl)-phenyl]-5-methyl-7-propyl-3H-imidazo[5,1-f]-[1,2,4]triazin-4-one;

2-{2-Ethoxy-5-[4-(2-hydroxyethyl)-piperazine-1-sulfonyl]-phenyl}-5-methyl-7-propyl-3H-imidazo[5,1-f][1,2,4]triazin-4-one;

2-{2-Ethoxy-5-[4-(2-hydroxyethyl)-piperazine-1-sulfonyl]-phenyl}-5-methyl-7-propyl-3H-imidazo[5,1-f][1,2,4]triazin-4-one hydrochloride;

2-{2-Ethoxy-5-[4-(3-hydroxypropyl)-piperazine-1-sulfonyl]-phenyl}-5-methyl-7-propyl-3H-imidazo[5,1-f][1,2,4]triazin-4-one;

N-Allyl-4-ethoxy-N-(2-hydroxyethyl)-3-(5-methyl-4-oxo-7-propyl-3,4-dihydro-imidazo[5,1-f]-[1,2,4]triazin-2-yl)benzenesulfonamide;

N-Ethyl-4-ethoxy-N-(2-hydroxyethyl)-3-(5-methyl-4-oxo-7-propyl-3,4-dihydro-imidazo[5,1-f]-[1,2,4]triazin-2-yl)benzenesulfonamide;

N,N-Diethyl-4-ethoxy-3-(5-methyl-4-oxo-7-propyl-3,4-dihydro-imidazo[5,1-f][1,2,4]triazin-2-yl)-benzenesulfonamide;

N-(2-Methoxyethyl)-3-(5-methyl-4-oxo-7-propyl-3,4-dihydro-imidazo[5,1-f][1,2,4]triazin-2-yl)-4-ethoxy-benzenesulfonamide;

N-(2-N,N-Dimethylethyl)-3-(5-methyl-4-oxo-7-propyl-3,4-dihydro-imidazo[5,1-f][1,2,4]triazin-2-yl)-4-ethoxy-benzenesulfonamide;

N-[3-(1-Morpholino)propyl]-3-(5-methyl-4-oxo-7-propyl-3,4-dihydro-imidazo[5,1-f][1,2,4]triazin-2-yl)-4-ethoxy-benzenesulfonamide;

N-{3-[1-(4-Methyl)piperazino]-propyl}-3-(5-methyl-4-oxo-7-propyl-3,4-dihydro-imidazo[5,1-f]-[1,2,4]triazin-2-yl)-4-ethoxy-benzenesulfonamide;

2-{2-Ethoxy-5-[4-(2-methoxyethyl)-piperazine-1-sulfonyl]-phenyl}-5-methyl-7-propyl-3H-imidazo[5,1-f][1,2,4]triazin-4-one;

2-{2-Ethoxy-5-[4-(2-N,N-dimethyl-ethyl)-piperazine-1-sulfonyl]-phenyl}-5-methyl-7-propyl-3H-imidazo[5,1-f][1,2,4]triazin-4-one;

2-{2-Ethoxy-5-[4-(3-N,N-dimethyl-propyl)-piperazine-1-sulfonyl]-phenyl}-5-methyl-7-propyl-3H-imidazo[5,1-f][1,2,4]triazin-4-one;

2-[2-Ethoxy-5-(4-dioxolano-piperidine-1-sulfonyl)-phenyl]-5-methyl-7-propyl-3H-imidazo[5,1-f]-[1,2,4]triazin-4-one;

2-[2-Ethoxy-5-(4-(5-methyl-4-furoxancarbonyl)-piperazine-1-sulfonyl)-phenyl]-5-methyl-7-propyl-3H-imidazo[5,1-f][1,2,4]triazin-4-one;

2-{2-Ethoxy-5-[4-acetyl-piperazine-1-sulfonyl]-phenyl}-5-methyl-7-propyl-3H-imidazo[5,1-f]-[1,2,4]triazin-4-one;

2-{2-Ethoxy-5-[4-formyl-piperazine-1-sulfonyl]-phenyl}-5-methyl-7-propyl-3H-imidazo[5,1-f]-[1,2,4]triazin-4-one;

2-[2-Ethoxy-5-(3-butylsydnonimine)-1-sulfonyl)-phenyl]-5-methyl-7-propyl-3H-imidazo[5,1-f]-[1,2,4]triazin-4-one;

5-Methyl-2-[5-(4-methyl-piperazine-1-sulfonyl)-2-propoxy-phenyl]-7-propyl-3H-imidazo[5,1-f]-[1,2,4]triazin-4-one;

5-Methyl-2-[5-(4-methyl-piperazine-1-sulfonyl)-2-propoxy-phenyl]-7-propyl-3H-imidazo[5,1-f]-[1,2,4]triazin-4-one hydrochloride;

2-[5-(4-Hydroxypiperidine-1-sulfonyl)-2-propoxy-phenyl]-5-methyl-7-propyl-3H-imidazo[5,1-f]-[1,2,4]triazin-4-one;

2-[5-(4-Hydroxymethylpiperidine-1-sulfonyl)-2-propoxy-phenyl]-5-methyl-7-propyl-3H-imidazo-[5,1-f][1,2,4]triazin-4-one;

2-{5-[4-(2-Hydroxyethyl)-piperazine-1-sulfonyl]-2-propoxy-phenyl}-5-methyl-7-propyl-3H-imidazo[5,1-f][1,2,4]triazin-4-one;

N-(1,1-Dioxotetrahydro-1λ⁶-thiophen-3-yl)-3-(5-methyl-4-oxo-7-propyl-3,4-dihydro-imidazo-[5,1-f][1,2,4]triazin-2-yl)-4-propoxy-benzenesulfonamide;

N-(2-Dimethylaminoethyl)-N-methyl-3-(5-methyl-4-oxo-7-propyl-3,4-dihydro-imidazo[5,1-f]-[1,2,4]triazin-2-yl)-4-propoxy-benzenesulfonamide;

3-(5-Methyl-4-oxo-7-propyl-3,4-dihydro-imidazo[5,1-f][1,2,4]triazin-2-yl)-N-(3-morpholin-4-yl-propyl)-4-propoxy-benzenesulfonamide;

N,N-Bis-(2-hydroxyethyl)-3-(5-methyl-4-oxo-7-propyl-3,4-dihydro-imidazo[5,1-f][1,2,4]triazin-2-yl)-4-propoxy-benzenesulfonamide;

N-(3-Hydroxybenzyl)-3-(5-methyl-4-oxo-7-propyl-3,4-dihydro-imidazo[5,1-f][1,2,4]triazin-2-yl)-4-propoxy-benzenesulfonamide;

N-Ethyl-N-(2-hydroxyethyl)-3-(5-methyl-4-oxo-7-propyl-3,4-dihydro-imidazo[5,1-f][1,2,4]triazin-2-yl)-4-propoxy-benzenesulfonamide;

N-(3-Ethoxypropyl)-3-(5-methyl-4-oxo-7-propyl-3,4-dihydro-imidazo[5,1-f][1,2,4]triazin-2-yl)-4-propoxy-benzenesulfonamide;

2-[5-(4-Hydroxypiperidine-1-sulfonyl)-2-propoxy-phenyl]-5-methyl-7-propyl-3H-imidazo[5,1-f]-[1,2,4]triazin-4-one;

3-(5-Methyl-4-oxo-7-propyl-3,4-dihydro-imidazo[5,1-f][1,2,4]triazin-2-yl)-4-propoxy-N-pyridin-4-yl-benzenesulfonamide;

N,N-Diethyl-3-(5-methyl-4-oxo-7-propyl-3,4-dihydro-imidazo[5,1-f][1,2,4]triazin-2-yl)-4-propoxy-benzenesulfonamide;

1-[3-(5-Methyl-4-oxo-7-propyl-3,4-dihydro-imidazo[5,1-f][1,2,4]triazin-2-yl)-4-propoxy-benzene-sulfonyl]piperidine-4-carboxylic acid;

5-Methyl-2-[5-(morpholine-4-sulfonyl)-2-propoxy-phenyl]-7-propyl-3H-imidazo[5,1-f][1,2,4]-triazin-4-one;

N-(2-Hydroxyethyl)-N-methyl-3-(5-methyl-4-oxo-7-propyl-3,4-dihydro-imidazo[5,1-f][1,2,4]-triazin-2-yl)-4-propoxy-benzenesulfonamide;

N-(2-Hydroxyethyl)-3-(5-methyl-4-oxo-7-propyl-3,4-dihydro-imidazo[5,1-f][1,2,4]triazin-2-yl)-4-propoxy-N-propyl-benzenesulfonamide;

N-[2-(3,4-Dimethoxyphenyl)-ethyl]-N-methyl-3-(5-methyl-4-oxo-7-propyl-3,4-dihydro-imidazo-[5,1-f][1,2,4]triazin-2-yl)-4-propoxy-benzenesulfonamide;

N-Allyl-N-(2-hydroxyethyl)-3-(5-methyl-4-oxo-7-propyl-3,4-dihydro-imidazo[5,1-f][1,2,4]triazin-2-yl)-4-propoxybenzenesulfonamide;

N-Allyl-N-cyclopentyl-3-(5-methyl-4-oxo-7-propyl-3,4-dihydro-imidazo[5,1-f][1,2,4]triazin-2-yl)-4-propoxybenzenesulfonamide;

N-Allyl-N-ethyl-3-(5-methyl-4-oxo-7-propyl-3,4-dihydro-imidazo[5,1-f][1,2,4]triazin-2-yl)-4-propoxybenzenesulfonamide;

2-[2-Ethoxy-4-methoxy-5-(4-methylpiperazine-1-sulfonyl)-phenyl]-5-methyl-7-propyl-3H-imidazo-[5,1-f][1,2,4]triazin-4-one;

2-{2-Ethoxy-5-[4-(2-hydroxyethyl)-piperazine-1-sulfonyl]-4-methoxy-phenyl}-5-methyl-7-propyl-3H-imidazo[5,1-f][1,2,4]triazin-4-one;

4-Ethoxy-N-ethyl-N-(2-hydroxyethyl)-2-methoxy-5-(5-methyl-4-oxo-7-propyl-3,4-dihydro-3,4-dihydro-imidazo[5,1-f][1,2,4]triazin-2-yl)-benzenesulfonamide;

4-Ethoxy-N-(4-ethoxyphenyl)-2-methoxy-5-(5-methyl-4-oxo-7-propyl-3,4-dihydro-imidazo[5,1-f]-[1,2,4]triazin-2-yl)-benzenesulfonamide;

4-Ethoxy-N-ethyl-N-(2-hydroxyethyl)-3-(5-ethyl-4-oxo-7-propyl-3,4-dihydroimidazo[5,1-f][1,2,4]-triazin-2-yl)benzenesulfonamide;

N-(2-Methoxyethyl)-3-(5-ethyl-4-oxo-7-propyl-3,4-dihydro-imidazo[5,1-f][1,2,4]triazin-2-yl)-4-ethoxybenzenesulfonamide;

N,N-Bis-(2-methoxyethyl)-3-(5-ethyl-4-oxo-7-propyl-3,4-dihydroimidazo[5,1-f][1,2,4]triazin-2-yl)-4-ethoxybenzenesulfonamide;

2-[5-(4-Hydroxypiperidine-1-sulfonyl)-2-ethoxyphenyl]-5-ethyl-7-propyl-3H-imidazo[5,1-f][1,2,4]triazin-4-one;

2-[5-(4-Hydroxymethylpiperidine-1-sulfonyl)-2-ethoxy-phenyl]-5-ethyl-7-propyl-3H-imidazo5,1-f][1,2,4]triazin-4-one;

2-{2-Ethoxy-5-[4-(2-hydroxyethyl)-piperazine-1-sulfonyl]-phenyl}-5-ethyl-7-propyl-3H-imidazo-[5,1-f][1,2,4]triazin-4-one;

2-[2-Ethoxy-5-(4-methylpiperazine-1-sulfonyl)-phenyl]-5-ethyl-7-propyl-3H-imidazo[5,1-f][1,2,4]-triazin-4-one;

2-[2-Ethoxy-5-(4-methylpiperazine-1-sulfonyl)-phenyl]-5-ethyl-7-propyl-3H-imidazo[5,1-f][1,2,4]-triazin-4-one hydrochloride;

3-(5-Ethyl-4-oxo-7-propyl-3,4-dihydro-imidazo[5,1-f][1,2,4]triazin-2-yl)-N-(3-morpholin-4-yl-propyl)-4-ethoxybenzenesulfonamide;

N-(2-Hydroxyethyl)-3-(5-ethyl-4-oxo-7-propyl-3,4-dihydro-imidazo[5,1-f][1,2,4]triazin-2-yl)-4-ethoxy-N-propyl-benzenesulfonamide;

2-[2-Ethoxy-5-(4-ethyl-piperazine-1-sulfonyl)-phenyl]-5-methyl-7-propyl-3H-imidazol[5,1-f]-[1,2,4]triazin-4-one hydrochloride trihydrate;

2-[2-Ethoxy-5-(4-ethyl-piperazine-1-sulfonyl)-phenyl]-5-methyl-7-propyl-3H-imidazo[5,1-f]-[1,2,4]triazin-4-one dihydrochloride.

Compounds particularly preferably used are listed in table A:

TABLE A
Structure

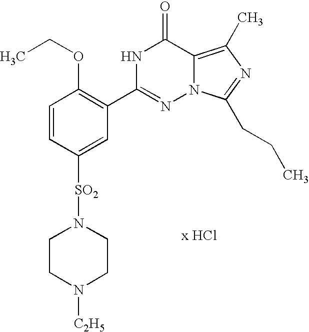

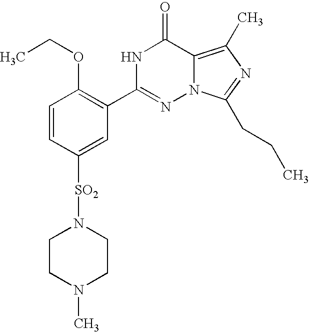

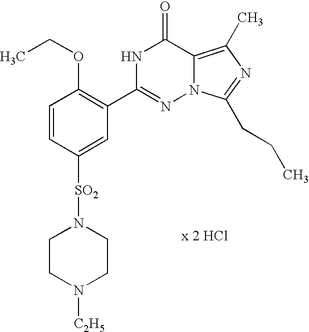

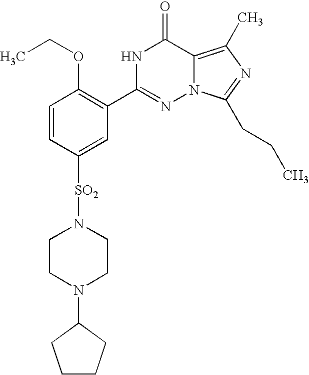

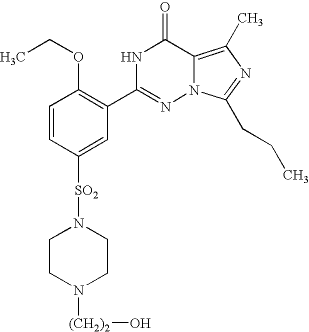

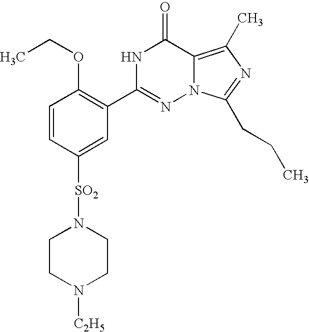

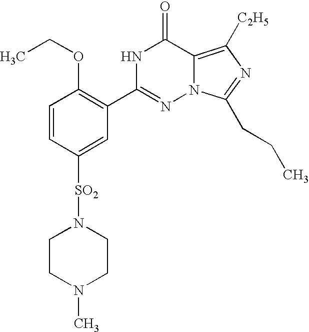

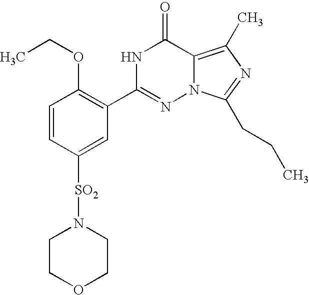

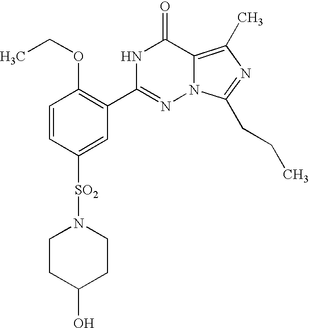

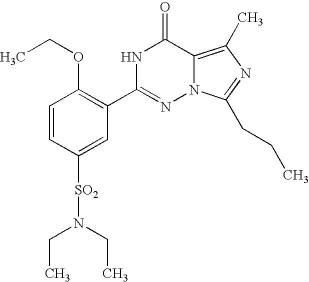

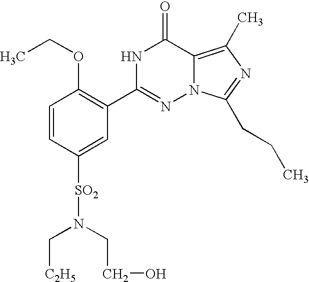

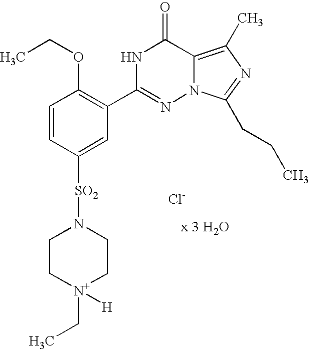

The compounds of the formulae (I) and (Ia) and of table A used according to the invention, and their preparation are described in WO 99/24433. The disclosure of WO 99/24433 is incorporated herein by reference.

A further embodiment of the invention relates to the use of the compounds of the general formulae (I) and (Ia) for manufacturing a medicament for the treatment of cardiac ischemia, for achieving or improving a preconditioning effect, for the treatment of an acute myocardial infarction and of reperfusion damage, specifically following a myocardial infarction, for the treatment of male infertility, of Raynaud's syndrome, of intermittent claudication, of Peyronie's disease, for the treatment of fibrotic disorders, of arteriosclerosis, for improving sperm motility, for the treatment of depression, leukemia (e.g. of chronic lymphocytic leukemia), for the treatment of priapism, for the treatment of platelet adhesion and aggregation associated with renal ischemia, for supporting and promoting liver regeneration following surgical resection of the liver or associated with liver cancer, for inhibiting the contraction of esophageal muscles (e.g. associated with nutcracker esophagus or esophagospasms), for the treatment of achalasia, premature labor, female infertility and dysmenorrhea, for the treatment of liver disorders such as, for example, cirrhosis of the liver, portal hypertension for the treatment of lupus, hypertensive systemic lupus erythematosus, scleroderma, for the treatment of multiple sclerosis, rheumatoid arthritis, allergy, autoimmune diseases, osteoporosis, cachexia, polycystic ovary syndrome, inflammatory bowel diseases such as, for example, Crohn's disease and ulcerative colitis, diabetic gangrene, diabetic arthropathy, diabetic glomerulosclerosis, diabetic dermatopathy, diabetic cataract, hyperlipidemia and dyslipidemia, for promoting growth and improving survival of oocytes, zygotes, embryos or fetuses, for increasing the weight of premature babies, for increasing milk production in mammals, specifically in humans, for the treatment of migraine, incontinence, acute and chronic renal failure, of glomerular disease, of nephritis, tubulointerstitial disorders, glomuleropathy, hair loss, pancreatitis, amnesia, disturbances of consciousness, autism, speech disturbances, Lennox syndrome and epilepsy.

The compounds used according to the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or otic route or as implant or stent.

The compounds used according to the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilisates or sterile powders.

Examples suitable for other administration routes are medicinal forms for inhalation (inter alia powder inhalators, nebulizers), nasal drops in solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

Oral or parenteral administration are preferred, especially oral and intravenous administration. Intravenous dosage is particularly preferred for example for the treatment of acute myocardial infarction and of reperfusion damage; incremental intravenous dosage is also possible here.

The compounds used according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking tastes and/or odors.

The present invention further relates to medicaments which comprise at least one compound used according to the invention, normally together with one or more inert, non-toxic, pharmaceutically suitable excipients, and to the use thereof for the aforementioned purposes.

It has generally proved advantageous on parenteral administration to administer amounts of about 0.001 to 10 mg/kg, preferably about 0.01 to 1 mg/kg, of body weight to achieve effective results. The amount on oral administration is about 0.01 to 100 mg/kg, preferably about 0.1 to 30 mg/kg, and very particularly preferably 0.1 to 10 mg/kg, of body weight.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The following exemplary embodiments illustrate the invention. The invention is not restricted to the examples.

Exemplary embodiment 1 is 2-[2-ethoxy-5-(4-methylpiperazine-1-sulfonyl)phenyl]-5-methyl-7-propyl-3H-imidazo[5,1-f][1,2,4]triazin-4-one; this compound is prepared in accordance with example 16 in WO 99/24433.

Exemplary embodiment 2 is 2-[2-ethoxy-5-(4-ethylpiperazine-1-sulfonyl)phenyl]-5-methyl-7-propyl-3H-imidazol[5,1-f][1,2,4]triazin-4-one hydrochloride trihydrate; this compound is prepared in accordance with example 336 in WO 99/24433.

The PDE- and PDE 5-inhibiting effect of the compounds used according to the invention can be determined as follows:

PDE 5 Inhibition Assay

The inhibitory effect is assayed by using the phosphodiesterase [³H] cGMP-SPA enzyme assay supplied by Amersham Life Science. The assay is carried out in accordance with the experimental protocol indicated by the manufacturer. Human recombinant PDE 5 which has been expressed in a bacculovirus system is used. The substance concentration at which the reaction rate is reduced by 50% is measured.

Exemplary embodiments 1 and 2 show IC₅₀ values respectively of 0.6 and 0.7 nM in this assay.

PDE Inhibition Assays

Recombinant PDE1C (GenBank/EMBL Accession Number: NM_{—}005020, Loughney et al. J. Biol. Chem. 1996, 271, 796-806), PDE2A (GenBank/EMBL Accession Number: NM_{—}002599, Rosman et al. Gene 1997, 191, 89-95), PDE3B (GenBank/EMBL Accession Number: NM_{—}000922, Miki et al. Genomics 1996, 36, 476-485), PDE4B (GenBank/EMBL Accession Number: NM_{—}002600, Obernolte et al. Gene. 1993, 129, 239-247), PDE5A (GenBank/EMBL Accession Number: NM_{—}001083, Loughney et al. Gene 1998, 216, 139-147), PDE7B (GenBank/EMBL Accession Number: NM_{—}018945, Hetman et al. Proc. Natl. Acad. Sci. U.S.A. 2000, 97, 472-476), PDE8A (GenBank/EMBL Accession Number: AF_{—}056490, Fisher et al. Biochem. Biophys. Res. Commun. 1998, 246, 570-577), PDE9A (Fisher et al., J. Biol. Chem, 1998, 273 (25): 15559-15564), PDE10A (GenBank/EMBL Accession Number: NM_{—}06661, Fujishige et al. J Biol Chem. 1999, 274, 18438-45), PDE11A (GenBank/EMBL Accession Number: NM_{—}016953, Fawcett et al. Proc. Natl. Acad. Sci. 2000, 97, 3702-3707) were expressed in Sf9 cells with the aid of the pFASTBAC baculovirus expression system (GibcoBRL).

The test substances are dissolved in 100% DMSO and serially diluted to determine their in vitro effect on PDE9A. Typically, serial dilutions from 200 μM to 1.6 μM are prepared (resulting final concentrations in the assay: 4 μM to 0.032 μM). 2 μL portions of the diluted substance solutions are introduced into the wells of microtiter plates (Isoplate; Wallac Inc., Atlanta, Ga.). Then 50 μL of a dilution of the PDE9A preparation described above are added. The dilution of the PDE9A preparation is chosen so that less than 70% of the substrate is converted during the subsequent incubation (typical dilution: 1:10 000; dilution buffer: 50 mM Tris/HCl pH 7.5, 8.3 mM MgCl₂, 1.7 mM EDTA, 0.2% BSA). The substrate, [8-³H]guanosine 3′,5′-cyclic phosphate (1 μCi/μL; Amersham Pharmacia Biotech., Piscataway, N.J.) is diluted 1:2000 with assay buffer (50 mM Tris/HCl pH 7.5, 8.3 mM MgCl₂, 1.7 mM EDTA) to a concentration of 0.0005 μCi/μL. The enzyme reaction is finally started by adding 50 μL (0.025 μCi) of the diluted substrate. The assay mixtures are incubated at room temperature for 60 min and the reaction is stopped by adding 25 μl of a PDE9A inhibitor (e.g. the compound from example 1 in WO/2004/026286, final concentration 10 μM) dissolved in assay buffer. Immediately thereafter, 25 μL of a suspension containing 18 mg/mL Yttrium Scintillation Proximity Beads (Amersham Pharmacia Biotech., Piscataway, N.J.) are added. The microtiter plates are sealed with a film and left to stand at room temperature for 60 min. The plates are then measured for 30 s per well in a Microbeta scintillation counter (Wallac Inc., Atlanta, Ga.). IC₅₀ values are determined from the graphical plot of the substance concentration versus the percentage inhibition.

The in vitro effect of test substances on recombinant PDE3B, PDE4B, PDE7B, PDE8A, PDE10A and PDE11A is determined in accordance with the assay protocol described above for PDE 9A with the following adaptations: [5′,8-³H]adenosine 3′,5′-cyclic phosphate (1 μCi/μL; Amersham Pharmacia Biotech., Piscataway, N.J.) is used as substrate. Addition of an inhibitor solution to stop the reaction is unnecessary. Instead, the incubation of substrate and PDE is followed immediately by addition of the yttrium scintillation proximity beads as described above and thus the reaction is stopped. To determine a corresponding effect on recombinant PDE1C, PDE2A and PDE5A, the protocol is additionally adapted as follows: with PDE1C, additionally 10⁻⁷ M calmodulin and 3 mM CaCl₂ are added to the reaction mixture. PDE2A is stimulated in the assay by adding 1 μM cGMP and is assayed with a BSA concentration of 0.01%. The substrate employed for PDE1C and PDE2A is [5′,8-³H]adenosine 3′,5′-cyclic phosphate (1 μCi/μL; Amersham Pharmacia Biotech., Piscataway, N.J.), and for PDE5A is [8-³H]guanosine 3′,5′-cyclic phosphate (1 μCi/μL; Amersham Pharmacia Biotech., Piscataway, N.J.).

1. The use of PDE 5 inhibitors for manufacturing a medicament for the treatment of cardiac ischemia, for achieving or improving a preconditioning effect, for the treatment of an acute myocardial infarction and of reperfusion damage, specifically following a myocardial infarction, for the treatment of male infertility, of Raynaud's syndrome, of intermittent claudication, of Peyronie's disease, for the treatment of fibrotic disorders, of arteriosclerosis, for improving sperm motility, for the treatment of depression, leukemia (e.g. of chronic lymphocytic leukemia), for the treatment of priapism, for the treatment of platelet adhesion and aggregation associated with renal ischemia, for supporting and promoting liver regeneration following surgical resection of the liver or associated with liver cancer, for inhibiting the contraction of esophageal muscles (e.g. associated with nutcracker esophagus or esophagospasms), for the treatment of achalasia, premature labor, female infertility and dysmenorrhea, for the treatment of liver disorders such as, for example, cirrhosis of the liver, portal hypertension, for the treatment of lupus, hypertensive systemic lupus erythematosus, scleroderma, for the treatment of multiple sclerosis, rheumatoid arthritis, allergy, autoimmune diseases, osteoporosis, cachexia, polycystic ovary syndrome, inflammatory bowel diseases such as, for example, Crohn's disease and ulcerative colitis, diabetic gangrene, diabetic arthropathy, diabetic glomerulosclerosis, diabetic dermatopathy, diabetic cataract, hyperlipidemia and dyslipidemia, for promoting growth and improving survival of oocytes, zygotes, embryos or fetuses, for increasing the weight of premature babies, for increasing milk production in mammals, specifically in humans, for the treatment of migraine, incontinence, acute and chronic renal failure, of glomerular disease, of nephritis, tubulointerstitial disorders, glomuleropathy, hair loss, pancreatitis, amnesia, disturbances of consciousness, autism, speech disturbances, Lennox syndrome and epilepsy.

2. The use as claimed in claim 1 of compounds of the formula (I) 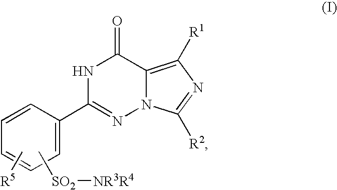
in which
R¹ is methyl or ethyl,
R² is ethyl or propyl,
R³ and R⁴ are identical or different and are a straight-chain or branched alkyl chain having up to 5 carbon atoms which is optionally substituted up to twice identically or differently by hydroxy or methoxy,
or
R³ and R⁴ together with the nitrogen atom form a piperidinyl, morpholinyl, thiomorpholinyl ring or a radical of the formula 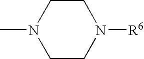
in which
R⁶ is hydrogen, formyl, acyl or alkoxycarbonyl having in each case up to 3 carbon atoms,
or
is straight-chain or branched alkyl having up to 3 carbon atoms which is optionally substituted once to twice, identically or differently, by hydroxy, carboxyl, straight-chain or branched alkoxy or alkoxycarbonyl having in each case up to 3 carbon atoms or by groups of the formulae —(CO)_{f}—NR⁷R⁸ or —P(O)(OR⁹)(OR¹⁰),
in which
f is a number 0 or 1,
R⁷ and R⁸ are identical or different and are hydrogen or methyl,
R⁹ and R¹⁰ are identical or different and are hydrogen, methyl or ethyl,
or
R⁶ is cyclopentyl,
and the heterocycles mentioned under R³ and R⁴ and formed together with the nitrogen atom are optionally substituted once to twice, identically or differently, optionally also geminally, by hydroxy, formyl, carboxyl, acyl or alkoxycarbonyl having in each case up to 3 carbon atoms or groups of the formulae —P(O)(OR¹¹)(OR¹²) or —(CO)ᵢ—NR¹³R¹⁴,
in which
R¹¹ and R¹² are identical or different and are hydrogen, methyl or ethyl,
i is a number 0 or 1,
and
R¹³ and R¹⁴ are identical or different and are hydrogen or methyl,
and/or the heterocycles mentioned under R³ and R⁴ and formed together with the nitrogen atom are optionally substituted by straight-chain or branched alkyl having up to 3 carbon atoms, which is optionally substituted once to twice, identically or differently, by hydroxy, carboxyl or by a radical of the formula —P(O)OR¹⁵OR¹⁶,
in which
R¹⁵ and R¹⁶ are identical or different and are hydrogen, methyl or ethyl,
and/or the heterocycles mentioned under R³ and R⁴ and formed together with the nitrogen atom are optionally substituted by N-linked piperidinyl or pyrrolidinyl,
and
R⁵ is ethoxy or propoxy,
and the salts and solvates thereof and the solvates of the salts.

3. The use as claimed in claim 2 of compounds of the formula (Ia) 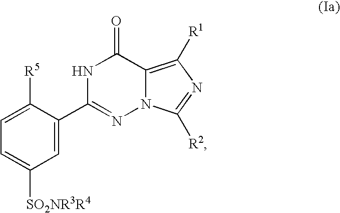
in which R¹, R², R³, R⁴ and R⁵ each have the meaning indicated in claim 2, and the salts and solvates thereof and the solvates of the salts.

4. The use as claimed in claim 3 of compounds which are selected from the group having the following structures:
Structure

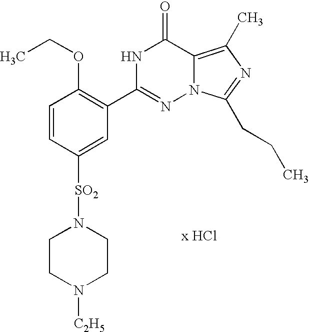

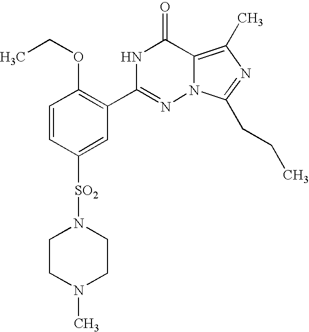

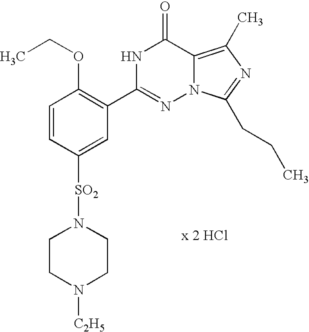

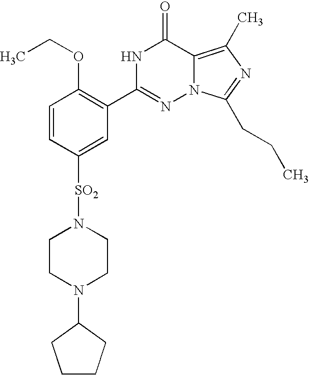

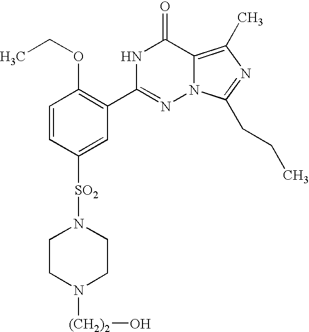

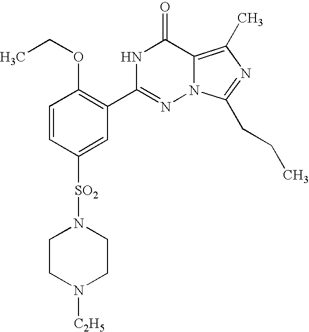

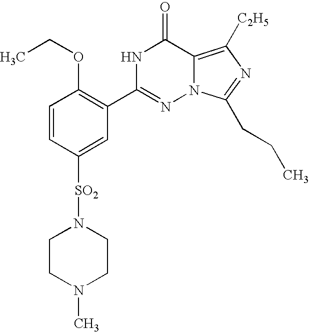

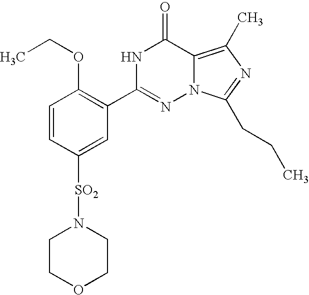

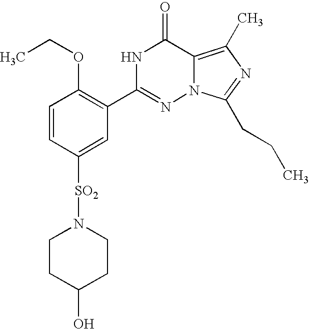

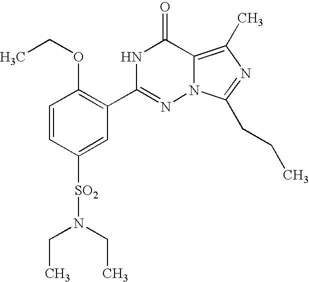

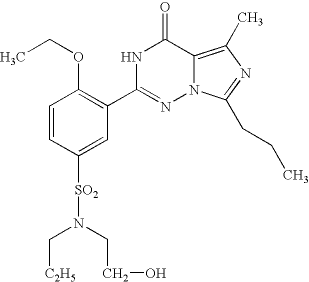

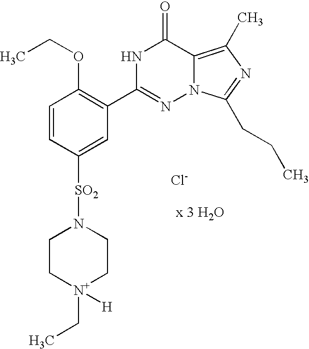

5. The use of the compounds as claimed in claim 4 for oral or intravenous treatment of acute myocardial infarction and reperfusion damage.
